SEQUENCE LISTING (13 sequences)

SEQ ID NO: 1 - DNA, 60 bp, Artificial Sequence
  Synthetic polynucleotide
caggagugagaugacaggaggccccaucttcatcaucactgagutatcacacaguggctg

SEQ ID NO: 2 - DNA, 61 bp, Artificial Sequence
  Synthetic polynucleotide
guctcgugggcucggagaugtgtauaagagacagacgcacagacaucctugcacatcucta

SEQ ID NO: 3 - DNA, 61 bp, Artificial Sequence
  Synthetic polynucleotide
caggagugagaugacaggagcacgacgutatagguattgagguccaautactugagacccu

SEQ ID NO: 4 - DNA, 59 bp, Artificial Sequence
  Synthetic polynucleotide
guctcgugggcucggagaugtgtauaagagacagcgaaacuacgucgaggacatcucaa

SEQ ID NO: 5 - DNA, 52 bp, Artificial Sequence
  Synthetic polynucleotide
caggagugagaugacaggaggcggagaggcagaggacaauactucatucccc

SEQ ID NO: 6 - DNA, 56 bp, Artificial Sequence
  Synthetic polynucleotide
guctcgugggcucggagaugtgtauaagagacaggcaugatggcauaggagucgcu

SEQ ID NO: 7 - DNA, 56 bp, Artificial Sequence
  Synthetic polynucleotide
caggagugagaugacaggagcguagtaacugacgagcuccaccutcctutaggcgg

SEQ ID NO: 8 - DNA, 58 bp, Artificial Sequence
  Synthetic polynucleotide
guctcgugggcucggagaugtgtauaagagacagccagcaagguaaagcautgucgca

SEQ ID NO: 9 - DNA, 6 bp, Artificial Sequence
  Synthetic polynucleotide
gaattc

SEQ ID NO: 10 - DNA, 9 bp, Artificial Sequence
  Synthetic polynucleotide
  misc_feature (4)..(6): n is a, c, g, or t
cagnnnctg

SEQ ID NO: 11 - DNA, 7 bp, Artificial Sequence
  Synthetic polynucleotide
  misc_feature (4)..(4): n is a, c, g, or t
cctnagg

SEQ ID NO: 12 - DNA, 6 bp, Artificial Sequence
  Synthetic polynucleotide
cccggg

SEQ ID NO: 13 - DNA, 11 bp, Artificial Sequence
  Synthetic polynucleotide
  misc_feature (3)..(9): n is a, c, g, or t
ccnnnnnnngg

(19) United States
(12) Patent Application Publication
(10) Pub. No.: US 2019/0185800 A1
(43) Pub. Date: Jun. 20, 2019

(54) MICROFLUIDIC SEQUENCING TECHNIQUES

(71) Applicant: President and Fellows of Harvard College, Cambridge, MA (US)

(72) Inventors: David A. Weitz, Bolton, MA (US); Huidan Zhang, Cambridge, MA (US); Nai Wen Cui, Cambridge, MA (US)

(73) Assignee: President and Fellows of Harvard College, Cambridge, MA (US)

(21) Appl. No.: 16/319,196

(22) PCT Filed: Jul. 20, 2017
(86) PCT No.: PCT/US2017/042998

§ 371 Date: Jan. 18, 2019

Related U.S. Application Data
(60) Provisional application No. 62/365,278, filed on Jul. 21, 2016.

(51) Int. Cl. C12M 3/06; C12Q 1/6858; C12M 1/00; C12Q 1/6806

(52) U.S. Cl. CPC C12M 23/16; C12Q 1/6858; C12M 47/06; C12Q 1/6806; C12Q 2535/122; C12Q 2525/131; C12Q 2563/159; C12Q 2565/629; C12Q 2521/301

(57) ABSTRACT

The present invention generally relates to microfluidics and, in some embodiments, to the determination of cells. In some aspects, primers able to introduce restriction sites into certain amplified nucleic acids are used. For example, the primers may introduce restriction sites into normal (wild-type) nucleic acids, but be unable to introduce restriction sites into mutant nucleic acids, e.g., due to a mismatch in the nucleic acid sequences caused by the mutant. After amplification, the nucleic acids may be exposed to a suitable restriction enzyme, which may cleave normal nucleic acids but not the mutant nucleic acids. In this way, mutant nucleic acids may be relatively quickly identified. In some embodiments, cells may be contained within microfluidic droplets and assayed to determine the mutant cells. In certain cases, for example, the nucleic acids may be amplified within droplets and attached to suitable tags, e.g., prior to breaking or merging the droplets and sequencing of the nucleic acids.

### RELATED APPLICATIONS

This application claims the benefit of U.S. Provisional Patent Application Ser. No. 62/365,278, filed Jul. 21, 2016, entitled “Microfluidic Sequencing Techniques,” by Weitz, et al., incorporated herein by reference in its entirety.

### FIELD

The present invention generally relates to microfluidics and, in some embodiments, to the determination of cells.

### BACKGROUND

Discovering the genetic roots of common diseases may be hard given the finding that large number of very rare genomic mutations may underline these diseases like cancer and schizophrenia, dimming the promise of personal genomics and the chances of quick medical payoffs from the human genome project. Until recently, rare mutations have been hard to catalog because of the difficulty of distinguishing an unusual mutation from an error in the DNA decoding process, which is roughly 0.1-1%. Now, however, a new generation of decoding machines allows each DNA unit in a genome to be examined 20 or more times, eliminating most errors. The coming challenge is how to study these mutations at a single-cell level to deeply understand the mechanism of many physiological and pathological processes, such as development and tumorigenesis due to their highly heterogeneous composition of cells. Another problem to overcome is how to sequence the amplicons only generated from mutant templates; otherwise the amplicons from wild-type templates will overwhelm sequencing capacity, which is not only very expensive, but also decreases sensitivity.

### SUMMARY

The present invention generally relates to microfluidics and, in some embodiments, to the determination of cells. The subject matter of the present invention involves, in some cases, interrelated products, alternative solutions to a particular problem, and/or a plurality of different uses of one or more systems and/or articles.

In one aspect, the present invention is generally directed to a method comprising lysing cells contained within microfluidic droplets to release nucleic acids, amplifying the released nucleic acids within the droplets using primers that introduce restriction sites during amplification to produce amplicons, bonding nucleic acid tags to at least some of the amplicons within the droplets, releasing the amplicons from the droplets, exposing the amplicons to a restriction enzyme, and sequencing the amplicons.

According to another aspect, the present invention is generally directed to a method comprising lysing cells contained within microfluidic droplets to release nucleic acids, amplifying the released nucleic acids within the droplets using primers that introduce restriction sites during amplification to produce amplicons, bonding nucleic acid tags to at least some of the amplicons within the droplets, releasing the amplicons from the droplets, exposing the amplicons to a restriction enzyme, and determining the amplicons not cleaved by the restriction enzyme.

In yet another aspect, the present invention is generally directed to a method comprising lysing cells contained within microfluidic droplets to release nucleic acids, amplifying the released nucleic acids within the droplets using primers that introduce restriction sites during amplification to produce amplicons, releasing the amplicons from the droplets, and exposing the amplicons to restriction enzymes.

In another aspect, the present invention encompasses methods of making one or more of the embodiments described herein. In still another aspect, the present invention encompasses methods of using one or more of the embodiments described herein.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is a schematic diagram illustrating amplification using various primers; and

FIG. 2 is a schematic diagram illustrating amplification within droplets.

### BRIEF DESCRIPTION OF THE SEQUENCES

SEQ ID NO: 1 is the forward primer BTK C481S, having the sequence

CAGGAGUGAGAUGACAGGAGGCCCCAUCTTCATCAUCACTGAGUtatcac
acagUGGCTG;

SEQ ID NO: 2 is the reverse primer BTK C481S, having the sequence

GUCTCGUGGGCUCGGAGAUGTGTAUAAGAGACAGacgCACAGACAUCCTU
GCACATCUCTA;

SEQ ID NO: 3 is the forward primer PLCG2 L845F, having the sequence

CAGGAGUGAGAUGACAGGAGCACGACGUTATAGGUATTGAGGUCCAAUta
ctuGAGACCCU;

SEQ ID NO: 4 is the reverse primer PLCG2 L8456F, having the sequence

GUCTCGUGGGCUCGGAGAUGTGTAUAAGAGACAGcgaAACUACGUCGAGG
ACATCUCAA;

SEQ ID NO: 5 is the forward primer PLCG2 R665W, having the sequence

CAGGAGUGAGAUGACAGGAG GCGGAGAGGCAGAGGACAauactucATUC
CCC;

SEQ ID NO: 6 is the reverse primer PLCG2 R665W, having the sequence

GUCTCGUGGGCUCGGAGAUGTGTAUAAGAGACAGgcaUGATGGCAUAGGA
GUCGCU;

SEQ ID NO: 7 is the forward primer PLCG2 S707Y, having the sequence

CAGGAGUGAGAUGACAGGAGCGUAGTAACUGACGAGCUCCACCUTCCTUT
AGGCGG;

SEQ ID NO: 8 is the reverse primer PLCG2 S707Y, having the sequence

GUCTCGUGGGCUCGGAGAUGTGTAUAAGAGACAGccaGCAAGGUAAAGCA
UTGUCGCA;

SEQ ID NO: 9 is the target sequence for EcoRI, GAATTC;

SEQ ID NO: 10 is the target sequence for AlwNI, CAGNNNCTG;

SEQ ID NO: 11 is the target sequence for Bsu36I, CCTNAGG;

SEQ ID NO: 12 is the target sequence for SmaI, CCCGGG; and

SEQ ID NO: 13 is the target sequence for BslI, CCNNNNNNNGG.

### DETAILED DESCRIPTION

The present invention generally relates to microfluidics and, in some embodiments, to the determination of cells. In some aspects, primers able to introduce restriction sites into certain amplified nucleic acids are used. For example, the primers may introduce restriction sites into normal (wild-type) nucleic acids, but be unable to introduce restriction sites into mutant nucleic acids, e.g., due to a mismatch in the nucleic acid sequences caused by the mutant. After amplification, the nucleic acids may be exposed to a suitable restriction enzyme, which may cleave normal nucleic acids but not the mutant nucleic acids. In this way, mutant nucleic acids may be relatively quickly identified. In some embodiments, cells may be contained within microfluidic droplets and assayed to determine the mutant cells. In certain cases, for example, the nucleic acids may be amplified within droplets and attached to suitable tags, e.g., prior to breaking or merging the droplets and sequencing of the nucleic acids.

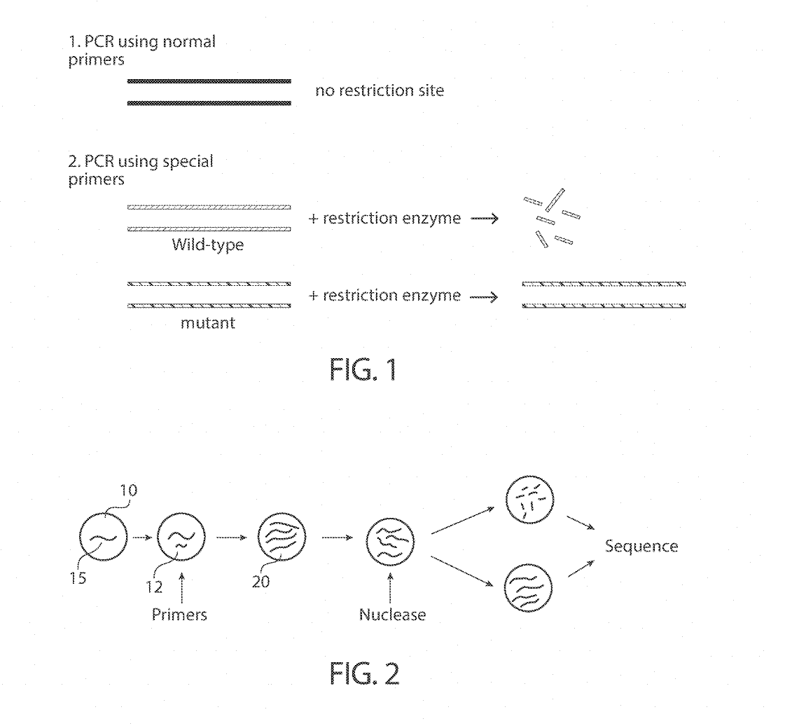

One aspect of the invention is generally directed to systems and methods for differentiating normal (wild-type) and mutant nucleic acids contained within droplets, such as microfluidic droplets. For example, in some cases, nucleic acids contained within droplets may be amplified using primers that are able to introduce restriction sites into wild-type sequences but are unable to introduce restriction sites into mutant sequences. See, e.g., FIG. 1 as an illustrative example. Upon exposure to a suitable restriction endonuclease, the wild-type sequences may be cleaved into shorter fragments while the mutant sequences lacking such restriction sites are not cleaved. Accordingly, by determining the nucleic acids that are present, e.g., their lengths and/or degree of amplification, the wild-type and mutant nucleic acids may be distinguished.

FIG. 2 illustrates a non-limiting schematic in accordance with another embodiment of the invention. In this embodiment, a plurality of droplets 10 is provided. The droplets may contain nucleic acids 15 arising from cells, or other sources. For instance, in some cases, droplets are prepared such that the droplets generally contain nucleic acids arising from a single source (e.g., a single lysed cell), or no nucleic acids. For instance, the droplets may be prepared such that the majority of droplets contains either one cell or no cell (e.g., at a density of less than 1 cell/droplet); causing lysis of the cells (if present) thereby causes each droplet to either contain nucleic acid from a single source (e.g., the lysed cell) or no nucleic acid. In this way, problems resulting from having the nucleic acids from multiple cells present within the same droplet may be minimized. (However, it should be understood that the invention is not so limited, and in other cases, it may be desirable to contain the nucleic acids from more than one source within a single droplet.)

In FIG. 2, the nucleic acids are exposed to suitable primers 12 for targeting a particular sequence, e.g., for amplification purposes. The primers 12 may be introduced into the droplets at any suitable time, e.g., when the droplet is formed or afterwards, and/or before or after the nucleic acids are introduced (e.g., before or after cell lysis within the droplets). The primers may be constructed so as to be able to introduce a restriction sequence during amplification, e.g., into the amplified nucleic acids that are produced (“amplicons,” for instance, amplicons 20 in FIG. 2). In addition, to facilitate amplification, suitable reagents may be added at any suitable point, e.g., before, during, or after primer exposure; these may include, for example, deoxyribonucleotides, polymerases, reverse transcriptases, and in some cases, restriction endonucleases.

During amplification, amplicons or amplified nucleic acids are produced during the amplification process. If the nucleic acid contains a target sequence, the amplification process may result in a restriction site being introduced into the amplified nucleic acids. For instance, the target sequence may be a wild-type sequence, or a specific mutated sequence. In some cases, however, the target sequence may have mutations in other, irrelevant portions of the nucleic acid such that the primer is still able to target and amplify the target sequence. Introduction of a suitable restriction site may be performed, for example, by adding a restriction site to the primer, e.g., to the 5′ end of the primer, or within a portion of the primer (e.g., within a middle portion). Many hundreds of potentially suitable restriction sequences and their associated restriction endonucleases are known to those of ordinary skill in the art, and many of those are commercially available. It should be noted that although this portion of the primer is mismatched to the target sequence and thus will not bind to it, the added restriction site will become incorporated into subsequent amplicons as the restriction site is also copied in addition to the target nucleic acid. In this way, the subsequent population of amplicons can include a restriction sequence that is introduced during the amplification process. It should be note that the length of the mismatched region (including the restriction site) may vary; for example, the mismatched region may be at least 5 nucleotides long, or other lengths as discussed herein.

Also shown in FIG. 2 is an example of DNA that is unable to sufficiently bind to the primer (lower branch). The DNA may arise from a different species (and thus be substantially different than the target sequence), or the DNA may be relatively similar but vary in such a way as to prevent or at least inhibit introduction of the restriction site to the subsequent amplicons. For example, the mutation may be in a portion such that when the mismatched primer is used to amplify the nucleic acid sequence, the mutation prevents the correct restriction sequence from being formed. As a non-limiting example, if the original sequence is ACCGGG and the primer introduces a mutation in the first position such that the correct restriction site is CCCGGG (e.g., a SmaI restriction site), then a mutated sequence such as ACAGGG (with a mutation in the third position) will be modified by the primer to the sequence CCAGGG, and thus, an enzyme such as SmaI will be unable to recognize and cleave it.

In this way, mutations or differences within the target nucleic acid may be distinguished by their inability to be cleaved when exposed to suitable restriction endonucleases. Thus, as is shown in FIG. 2, after amplification, a restriction endonuclease may be introduced into the droplets, then the droplets may be analyzed for nucleic acids; those with the correct target nucleic acid may be present as fragments (or significantly shorter lengths) than those droplets not having the correct target nucleic acid. If no nucleic acids are present within a droplet, and/or the nucleic acids are substantially different, e.g., from a contaminating species, then no amplification would occur and the droplets may appear to be relatively free of any amplified nucleic acid sequences.

The above discussion is a non-limiting example of one embodiment of the present invention that can be used to introduce restriction sites into certain amplified nucleic acids. However, other embodiments are also possible. Accordingly, more generally, various aspects of the invention are directed to various systems and methods for determination of certain population of cells, e.g., by amplifying their DNA within droplets.

In one aspect, the present invention is generally directed to systems and methods for determining whether a nucleic acid contains or does not contain a target sequence by exposing the target sequence to a primer able to add a restriction site to sequences amplified from the target sequence (i.e., amplicons), then exposing the amplicons to a restriction endonuclease able to target the restriction site. If the restriction site is present, then the restriction endonuclease may cleave or fragment the amplicons. However, if the restriction site is not present, then the restriction endonuclease may be unable to cleave or fragment the amplicons. In this way, nucleic acids that do or do not contain the target sequence can be readily distinguished from each other. In some cases, the target sequence may be a wild-type sequence, which may be distinguished from certain mutants of the target sequence, e.g., having a mutation in certain parts of the sequence. In other embodiments, however, the target sequence may be a mutant or other sequence, or the target sequence may be from a first species and the other sequences may be from other species. The target sequence may be DNA, e.g., genomic DNA, or in some cases, the target sequence may be RNA.

In some cases, the nucleic acid may arise from cells. For example, in some cases, cells may be encapsulated within droplets, then lysed within the droplets to release nucleic acids into the droplets. In this way, nucleic acids from different cells may be kept isolated from each other, e.g., as they are contained within different droplets. In some cases, as discussed below, the cells may be encapsulated within droplets at relatively low densities, e.g., less than 1 cell/droplet, to minimize the number of droplets containing two or more cells. However, it should be understood that in other embodiments, the nucleic acids may arise from other sources, not necessarily only lysed cells. For example, the nucleic acids may be synthetically prepared, or purified from cells using other techniques prior to being introduced into droplets. In addition, in some cases, more than one cell may be desirably present within some or all of the droplets.

Non-limiting examples of cells that may be determined include cancer cells (or cells suspected of being cancerous), normal cells, foreign cells (e.g., bacteria, fungi, pathogens, etc.), viruses, or the like. In some cases, the cell may be an infected cell, e.g., a cell infected with a bacterium, a virus, a fungus, a pathogen, or the like. The cells may arise from any suitable species. For example, the cells may include a eukaryotic cell, an animal cell, a plant cell, a bacterium or other single-cell organism, etc. If the cell is an animal cell, the cell may be, for example, an invertebrate cell (e.g., a cell from a fruit fly), a fish cell (e.g., a zebrafish cell), an amphibian cell (e.g., a frog cell), a reptile cell, a bird cell, or a human or non-human mammal, such as a monkey, ape, cow, sheep, goat, horse, rabbit, pig, mouse, rat, guinea pig, dog, cat, etc. If the cell is from a multicellular organism, the cell may be from any part of the organism. For instance, if the cell is from an animal, the cell may be an immune cell, a cardiac cell, a fibroblast, a keratinocyte, a heptaocyte, a chondracyte, a neural cell, an osteocyte, an osteoblast, a muscle cell, a blood cell, an endothelial cell, or the like. In some cases, the cell is genetically engineered. In some cases, at least some of the cells arise from dissociated tissue or organs.

As mentioned, in some embodiments, the droplets can be loaded such that, on the average, each droplet has less than 1 cell in it. For example, the average loading rate may be less than about 1 cell/droplet, less than about 0.9 cells/droplet, less than about 0.8 cells/droplet, less than about 0.7 cells/droplet, less than about 0.6 cells/droplet, less than about 0.5 cells/droplet, less than about 0.4 cells/droplet, less than about 0.3 cells/droplet, less than about 0.2 cells/droplet, less than about 0.1 cells/droplet, less than about 0.05 cells/droplet, less than about 0.03 cells/droplet, less than about 0.02 cells/droplet, or less than about 0.01 cells/droplet. In some cases, lower cell loading rates may be chosen to minimize the probability that a droplet will be produced having two or more cells in it. Thus, for example, at least about 50%, at least about 60%, at least about 70%, at least about 80%, at least about 90%, at least about 95%, at least about 97%, at least about 98%, or at least about 99% of the droplets may contain either no cell or only one cell. Those of ordinary skill in the art will be aware of suitable techniques for loading a cell into a droplet. In addition, in certain embodiments, it may be desired to encapsulate cells at higher rates, e.g., greater than 1 cell/droplet, greater than 2 cells/droplets, etc.

Any suitable method may be chosen to create droplets, and a wide variety of different techniques for forming droplets will be known to those of ordinary skill in the art. For example, a junction of channels may be used to create the droplets. The junction may be, for instance, a T-junction, a Y-junction, a channel-within-a-channel junction (e.g., in a coaxial arrangement, or comprising an inner channel and an outer channel surrounding at least a portion of the inner channel), a cross (or “X”) junction, a flow-focusing junction, or any other suitable junction for creating droplets. See, for example, International Patent Application No. PCT/US2004/010903, filed Apr. 9, 2004, entitled “Formation and Control of Fluidic Species,” by Link, et al., published as WO 2004/091763 on Oct. 28, 2004, or International Patent Application No. PCT/US2003/020542, filed Jun. 30, 2003, entitled “Method and Apparatus for Fluid Dispersion,” by Stone, et al., published as WO 2004/002627 on Jan. 8, 2004, each of which is incorporated herein by reference in its entirety. In some embodiments, the junction may be configured and arranged to produce substantially monodisperse droplets. The droplets may also be created on the fluidic device, and/or the droplets may be created separately then brought to the device.

In one set of embodiments, if cells are present, the cells may be lysed within the droplets, e.g., to release DNA and/or RNA from the cell, and/or to produce a cell lysate within the droplet. For instance, the cells may be lysed via exposure to a lysing chemical or a cell lysis reagent (e.g., a surfactant such as Triton-X or SDS, an enzyme such as lysozyme, lysostaphin, zymolase, cellulase, mutanolysin, glycanases, proteases, mannase, proteinase K, etc.), or a physical condition (e.g., ultrasound, ultraviolet light, mechanical agitation, etc.). If a lysing chemical is used, the lysing chemical may be introduced into the droplet after formation of the droplet, e.g., through picoinjection or other methods such as those discussed in U.S. patent application Ser. No. 13/379,782, filed Dec. 21, 2011, entitled “Fluid Injection,” published as U.S. Pat. Apl. Pub. No. 2012/0132288 on May 31, 2012, incorporated herein by reference in its entirety, through fusion of the droplets with droplets containing the chemical or enzyme, or through other techniques known to those of ordinary skill in the art. In some cases, lysing a cell will cause the cell to release its contents, e.g., genomic DNA, various RNAs, etc. In some embodiments, some of the cellular nucleic acids may also be joined to one or more oligonucleotides contained within the droplet, e.g., as discussed herein.

In certain embodiments, a primer may be used that is able to introduce restriction sites into certain amplified nucleic acids. For instance, the primer may be designed to be able to bind a target sequence, and upon amplification, add a certain sequence (e.g., a sequence including a restriction site) into the amplified nucleic acids (or amplicons) that are produced during the amplification process. The target sequence may be a wild-type sequence, or a specific mutated sequence. In some cases, the target sequence may have mutations in other, irrelevant portions of the nucleic acid such that the primer is still able to target and amplify the target sequence. Introduction of a suitable restriction site may be performed, for example, by adding a restriction site to the primer, e.g., to the 5′ end of the primer, or internally of the primer. As discussed, if the nucleic acid contains a desired target sequence, amplification may result in the restriction site being introduced into the amplified nucleic acids (i.e., amplicons) that are produced. Thus, in some cases, the primer may include a first portion able to bind to or interact with the target sequence, and a second portion that is unable to bind to or interact with the target sequence. In some cases, the first portion is at least 50%, at least 75%, at least 80%, at least 85%, at least 90%, or at least 95% complementary to a portion of the target sequence.

The second portion may be incorporated into the amplicons during amplification of the target sequence. If the second portion includes a restriction site, then the subsequent amplicons may also contain the restriction site. In some cases, selectivity may be achieved through interaction of the first portion with the target sequence; e.g., if the target sequence is not present (e.g., due to a mutation or due to the lack of the presence of a nucleic acid containing the target sequence), then no amplification using the primer can occur.

The second portion that is added to the amplicon may have any suitable length. For example, the second portion may have a length of at least 5 nucleotides, at least 10 nucleotides, at least 15 nucleotides, at least 20 nucleotides, at least 25 nucleotides, at least 30 nucleotides, at least 35 nucleotides, at least 40 nucleotides, at least 45 nucleotides, or at least 50 nucleotides. In some cases, the second portion may also have a maximum length of no more than 100 nucleotides, no more than 75 nucleotides, no more than 50 nucleotides, etc. The restriction site may occur at any suitable location (or more than one location, in some cases) within the second portion.

The target sequence may be any suitable sequence, for example, one in which it is desired to distinguish the target sequence from other sequences. For instance, the target sequence may be a wild-type sequence, or the target sequence may have one or more mutations. In some cases, the target sequence may have mutations in other, irrelevant portions of the nucleic acid such that the primer is still able to target and amplify the target sequence.

Those of ordinary skill in the art will know of hundreds of potentially suitable restriction sequences, and their associated restriction endonucleases. Many such restriction endonucleases are readily available commercially. Non-limiting examples include those discussed herein, such as EcoRI, AlwNI, Bsu36I, SmaI, BslI, or the like.

In some cases, the nucleic acids may be amplified, e.g., within the droplets, for example, by including suitable reagents specific to the amplification method. Examples of amplification methods known to those of ordinary skill in the art include, but are not limited to, polymerase chain reaction (PCR), reverse transcriptase (RT) PCR amplification, in vitro transcription amplification (IVT), multiple displacement amplification (MDA), or quantitative real-time PCR (qPCR). See also U.S. Pat. Apl. Ser. No. 61/981,108, 62/072,944, or 62/133,140, or U.S. Pat. Apl. Pub. No. 2010/0136544, 2014/0199730, or 2014/0199731, each incorporated by reference in its entirety.

In one set of embodiments, for example, PCR or nucleic acid amplification may be performed within the droplets. For example, the droplets may contain a primer (such as those discussed herein), a polymerase (such as Taq polymerase), and DNA nucleotides, and the droplets may be processed (e.g., via repeated heated and cooling) to amplify the nucleic acid within the droplets. The polymerase, primers, and nucleotides may be added at any suitable point, and may be added sequentially and/or simultaneously, using any suitable technique (e.g., using droplet fusion or injection techniques). For instance, a droplet may contain a suitable polymerase and DNA nucleotides, which is fused to the droplet to allow amplification to occur. Those of ordinary skill in the art will be aware of suitable PCR techniques and variations, such as assembly PCR or polymerase cycling assembly, which may be used in some embodiments to produce an amplified nucleic acid.

As mentioned, in some cases, amplified nucleic acids containing a target sequence may be distinguished from those not containing the target sequence through exposure to restriction endonucleases, which are enzymes able to cleave nucleic acids at a specific site (a restriction site), if present. If the restriction site is not present, then the restriction endonuclease is generally incapable of cleaving the nucleic acid. In this way, target sequences containing the restriction site (added as discussed above) may be distinguished from non-target sequences that do not contain the restriction site. Non-limiting examples of restriction endonucleases include EcoRI, EcoRII, BamHI, HindIII, TaqI, EcoP15, AlwNI, Bsu36I, BslI, and SmaI, etc. Many such restriction endonucleases are commercially available. Those of ordinary skill in the art will be aware of restriction endonucleases and their corresponding restriction sites.

In some cases, the nucleic acids within the droplets may be determined or distinguished in some fashion. For instance, amplicons within the droplets may be sequenced. In some embodiments, the droplets may be burst or broken to release their contents, and nucleic acids from different droplets combined together for sequencing purposes. In some cases, however, the nucleic acids within the various droplets may be uniquely identified or “tagged” prior to release from the droplets, e.g., so as to be able to subsequently distinguish nucleic acids arising from different droplets. One non-limiting example of such a technique is to label the nucleic acids with unique oligonucleotides or “barcodes” prior to their release from the droplets.

For instance, in some embodiments, the nucleic acids from the cell (e.g., DNA and/or RNA) may be bonded to one or more oligonucleotides, e.g., covalently, through primer extension, through ligation, or the like, prior to release from the droplets. Any of a wide variety of different techniques may be used, and those of ordinary skill in the art will be aware of many such techniques. The exact joining technique used is not necessarily critical, and can vary between embodiments.

For instance, in certain embodiments, the nucleic acids may be joined with the oligonucleotides using ligases. Non-limiting examples of ligases include DNA ligases such as DNA Ligase I, DNA Ligase II, DNA Ligase III, DNA Ligase IV, T4 DNA ligase, T7 DNA ligase, T3 DNA Ligase, E. coli DNA Ligase, Taq DNA Ligase, or the like. Many such ligases may be purchased commercially. As additional examples, in some embodiments, two or more nucleic acids may be ligated together using annealing or a primer extension method.

In yet another set of embodiments, the nucleic acids may be joined with the oligonucleotides and/or amplified using PCR (polymerase chain reaction) or other suitable amplification techniques, including any of those recited herein. Typically, in PCR reactions, the nucleic acids are heated to cause dissociation of the nucleic acids into single strands, and a heat-stable DNA polymerase (such as Taq polymerase) is used to amplify the nucleic acid. This process is often repeated multiple times to amplify the nucleic acids.

In some embodiments, the oligonucleotides may comprise a “barcode” or a unique sequence. The sequence may be selected such that some or all of the oligonucleotides have the unique sequence (or combination of sequences that is unique), but other oligonucleotides (e.g., in other droplets) do not have the unique sequence or combination of sequences. Thus, for example, the sequences may be used to uniquely identify or distinguish a droplet, or nucleic acid contained arising from the droplet (e.g., from a lysed cell) from other droplets, or other nucleic acids (e.g., released from other cells) arising from other droplets, or released after the droplets are broken or dispersed.

The oligonucleotide sequences may be of any suitable length. The length of the oligonucleotide sequence is not critical, and may be of any length sufficient to distinguish the oligonucleotide sequence from other oligonucleotide sequences. One, two, or more such distinguishing “barcode” sequence may be present in an oligonucleotide, as discussed above. A barcode sequence can have, for instance, a length of 5, 6, 7, 8, 9, 10, 11, 12, 13, 14, 15, 16, 17, 18, 19, 20, 21, 22, 23, 24, or 25 nt. More than 25 nucleotides may also be present in some cases.

In some cases, the unique oligonucleotide or barcode sequences may be taken from a “pool” of potential sequences. If more than one barcode sequence is present in an oligonucleotide, the barcode sequences may be taken from the same, or different pools of potential barcode sequences. The pool of sequences may be selected using any suitable technique, e.g., randomly, or such that the sequences allow for error detection and/or correction, for example, by being separated by a certain distance (e.g., Hamming distance) such that errors in reading of the barcode sequence can be detected, and in some cases, corrected. The pool may have any number of potential barcode sequences, e.g., at least 100, at least 300, at least 500, at least 1,000, at least 3,000, at least 5,000, at least 10,000, at least 30,000, at least 50,000, at least 100,000, at least 300,000, at least 500,000, or at least 1,000,000 barcode sequences.

In some embodiments of the present invention, the barcoded nucleic acids attached to particles or microspheres, e.g., for delivery to droplets. For example, one set of embodiments is generally directed to particles or microspheres carrying nucleic acid fragments (each encoding a barcode, a primer, and/or other sequences possibly used for capture, amplification and/or sequencing of nucleic acids). Microspheres may include a hydrogel particle (polyacrylamide, agarose, etc.), or a colloidal particle (polystyrene, magnetic or polymer particle, etc.), having dimensions such as those described herein. The microspheres may be porous in some embodiments. Other suitable particles or microspheres that can be used are discussed in more detail herein.

The preparation of particles or microspheres, in some cases, may rely on the covalent attachment or other techniques of incorporation of an initial DNA oligonucleotide to the particles or microspheres, followed by enzymatic extension of each oligonucleotide by one or more barcodes selected, e.g., at random, from a pre-defined pool. The final number of possible unique barcodes may depend in some cases on the size of the pre-defined barcode pool and/or on the number of extension steps. For example, using a pool of 384 pre-defined barcodes and 2 extension steps, each particle or microsphere carries one of 384²=147,456 possible barcodes; using 3 extension steps, each particle or microsphere carries one of 384³=56,623,104 possible barcodes; and so on. Other numbers of steps may also be used in some cases; in addition, each pool may have various numbers of pre-defined barcodes (not just 384), and the pools may have the same or different numbers of pre-defined barcodes. The pools may include the same and/or different sequences.

Accordingly, in some embodiments, the possible barcodes that are used are formed from one or more separate “pools” of barcode elements that are then joined together to produce the final barcode, e.g., using a split-and-pool approach. A pool may contain, for example, at least about 300, at least about 500, at least about 1,000, at least about 3,000, at least about 5,000, or at least about 10,000 distinguishable barcodes. For example, a first pool may contain x₁ elements and a second pool may contain x₂ elements; forming a barcode containing an element from the first pool and an element from the second pool may yield, e.g., x₁x₂ possible barcodes that could be used. It should be noted that x₁ and x₂ may or may not be equal. This process can be repeated any number of times; for example, the barcode may include elements from a first pool, a second pool, and a third pool (e.g., producing x₁x₂x₃ possible barcodes), or from a first pool, a second pool, a third pool, and a fourth pool (e.g., producing x₁x₂x₃x₄ possible barcodes), etc. There may also be 5, 6, 7, 8, or any other suitable number of pools. Accordingly, due to the potential number of combinations, even a relatively small number of barcode elements can be used to produce a much larger number of distinguishable barcodes.

In some cases, such use of multiple pools, in combination, may be used to create substantially large numbers of useable barcodes, without having to separately prepare and synthesize large numbers of barcodes individually. For example, in many prior art systems, requiring 100 or 1,000 barcodes would require the individual synthesis of 100 or 1,000 barcodes. However, if larger numbers of barcodes are needed, e.g., for larger numbers of cells to be studied, then correspondingly larger numbers of barcodes would need to be synthesized. Such systems become impractical and unworkable at larger numbers, such as 10,000, 100,000, or 1,000,000 barcodes. However, by using separate “pools” of barcodes, larger numbers of barcodes can be achieved without necessarily requiring each barcode to be individually synthesized. As a non-limiting example, a first pool of 1,000 distinguishable barcodes (or any other suitable number) and a second pool of 1,000 distinguishable barcodes can be synthesized, requiring the synthesis of 2,000 barcodes (or only 1,000 if the barcodes are re-used in each pool), yet they may be combined to produce 1,000×1,000=1,000,000 distinguishable barcodes, e.g., where each distinguishable barcode comprises a first barcode taken from the first pool and a second barcode taken from the second pool. Using 3, 4, or more pools to assemble the barcode may result in even larger numbers of barcodes that may be prepared, without substantially increasing the total number of distinguishable barcodes that would need to be synthesized.

The oligonucleotide may be of any suitable length or comprise any suitable number of nucleotides. The oligonucleotide may comprise DNA, RNA, and/or other nucleic acids such as PNA, and/or combinations of these and/or other nucleic acids. In some cases, the oligonucleotide is single stranded, although it may be double stranded in other cases. For example, the oligonucleotide may have a length of at least about 10 nt, at least about 30 nt, at least about 50 nt, at least about 100 nt, at least about 300 nt, at least about 500 nt, at least about 1000 nt, at least about 3000 nt, at least about 5000 nt, at least about 10,000 nt, etc. In some cases, the oligonucleotide may have a length of no more than about 10,000 nt, no more than about 5000 nt, no more than about 3000 nt, no more than about 1000 nt, no more than about 500 nt, no more than about 300 nt, no more than about 100 nt, no more than about 50 nt, etc. Combinations of any of these are also possible, e.g., the oligonucleotide may be between about 10 nt and about 100 nt. The length of the oligonucleotide is not critical, and a variety of lengths may be used in various embodiments.

The oligonucleotide may also contain a variety of sequences. For example, the oligonucleotide may contain one or more primer sequences, one or more unique or “barcode” sequences as discussed herein, one or more promoter sequences, one or more spacer sequences, or the like. The oligonucleotide may also contain, in some embodiments one or more cleavable spacers, e.g., photocleavable linker. The oligonucleotide may in some embodiments be attached to a particle chemically (e.g., via a linker) or physically (e.g., without necessarily requiring a linker), e.g., such that the oligonucleotides can be removed from the particle via cleavage. Other examples include portions that may be used to increase the bulk (or length) of the oligonucleotides (e.g., using specific sequences or nonsense sequences), to facilitate handling (for example, an oligonucleotide may include a poly-A tail), to increase selectivity of binding (e.g., as discussed below), to facilitate recognition by an enzyme (e.g., a suitable ligase), to facilitate identification, or the like. Examples of these and/or other sequences are described in further detail herein. In some cases, the oligonucleotide may contain one or more promoter sequences, e.g., to allow for production of the oligonucleotide, to allow for enzymatic amplification, or the like.

In some cases, the oligonucleotide may contain nonsense or random sequences, e.g., to increase the mass or size of the oligonucleotide. The random sequence can be of any suitable length, and there may be one or more than one present. As non-limiting examples, the random sequence may have a length of 10 to 40, 10 to 30, 10 to 20, 25 to 50, 15 to 40, 15 to 30, 20 to 50, 20 to 40, or 20 to 30 nucleotides.

In some cases, the oligonucleotide may comprise one or more sequences able to specifically bind a gene or other entity. For example, in one set of embodiments, the oligonucleotide may comprise a sequence able to recognize mRNA, e.g., one containing a poly-T sequence (e.g., having several T's in a row, e.g., 4, 5, 6, 7, 8, or more T's).

In one set of embodiments, the oligonucleotide may contain one or more cleavable linkers, e.g., that can be cleaved upon application of a suitable stimulus. For example, the cleavable sequence may be a photocleavable linker that can be cleaved by applying light or a suitable chemical or enzyme. In some cases, for example, a plurality of particles (containing oligonucleotides on their surfaces) may be prepared and added to droplets, e.g., such that, on average, each droplet contains one particle, or less (or more) in some cases. After being added to the droplet, the oligonucleotides may be cleaved from the particles, e.g., using light or other suitable cleavage techniques, to allow the oligonucleotides to become present in solution, i.e., within the interior of the droplet. In such fashion, oligonucleotides can be easily loaded into droplets by loading of the particles into the droplets, then cleaved off to allow the oligonucleotides to be in solution, e.g., to interact with nucleotides or other species, such as is discussed herein.

A variety of techniques may be used for preparing oligonucleotides such as those discussed herein. These may be prepared in bulk and/or in one or more droplets, such as microfluidic droplets. In some cases, the oligonucleotides may be prepared in droplets, e.g., to ensure that the barcodes and/or oligonucleotides within each droplet are unique. In addition, in some embodiments, particles may be prepared containing oligonucleotides with various barcodes in separate droplets, and the particles may then be given or sold to a user who then adds the nucleic acids to the oligonucleotides, e.g., as described above.

In some cases, an oligonucleotide comprising DNA and/or other nucleic acids may be attached to particles and delivered to the droplets. In some cases, the oligonucleotides are attached to particles to control their delivery into droplets, e.g., such that a droplet will typically have at most one particle in it. In some cases, upon delivery into a droplet, the oligonucleotide may be removed from the particle, e.g., by cleavage, by degrading the particle, etc. However, it should be understood that in other embodiments, a droplet may contain 2, 3, or any other number of particles, which may have oligonucleotides that are the same or different.

In some embodiments, the barcoded oligonucleotides introduced into droplets using particles or microspheres can be cleaved therefrom by, e.g., light, chemical, enzymatic or other techniques, e.g., to improve the efficiency of priming enzymatic reactions in droplets. However, the cleavage of the primers can be performed at any step or point, and can be defined by the user in some cases. Such cleavage may be particularly important in certain circumstances and/or conditions; for example, some fraction of RNA and DNA molecules in single cells might be very large, or might be associated in complexes and therefore will not diffuse efficiently to the surface or interior of the particle or microsphere. However, in other embodiments, cleavage is not essential.

Any suitable method may be used to attach the oligonucleotide to the particle. The exact method of attachment is not critical, and may be, for instance, chemical or physical. For example, the oligonucleotide may be covalently bonded to the particle via a biotin-steptavidin linkage, an amino linkage, or an acrylic phosphoramidite linkage. In another set of embodiments, the oligonucleotide may be incorporated into the particle, e.g., physically, where the oligonucleotide may be released by altering the particle. Thus, in some cases, the oligonucleotide need not have a cleavable linkage. For instance, in one set of embodiments, an oligonucleotide may be incorporated into particle, such as an agarose particle, upon formation of the particle. Upon degradation of the particle (for example, by heating the particle until it begins to soften, degrade, or liquefy), the oligonucleotide may be released from the particle.

The particle is a microparticle in certain embodiments. The particle may be of any of a wide variety of types; as discussed, the particle may be used to introduce a particular oligonucleotide into a droplet, and any suitable particle to which oligonucleotides can associate with (e.g., physically or chemically) may be used. The exact form of the particle is not critical. The particle may be spherical or non-spherical, and may be formed of any suitable material. In some cases, a plurality of particles is used, which have substantially the same composition and/or substantially the same average diameter. The “average diameter” of a plurality or series of particles is the arithmetic average of the average diameters of each of the particles. Those of ordinary skill in the art will be able to determine the average diameter (or other characteristic dimension) of a plurality or series of particles, for example, using laser light scattering, microscopic examination, or other known techniques. The average diameter of a single particle, in a non-spherical particle, is the diameter of a perfect sphere having the same volume as the non-spherical particle. The average diameter of a particle (and/or of a plurality or series of particles) may be, for example, less than about 1 mm, less than about 500 micrometers, less than about 200 micrometers, less than about 100 micrometers, less than about 75 micrometers, less than about 50 micrometers, less than about 25 micrometers, less than about 10 micrometers, or less than about 5 micrometers in some cases. The average diameter may also be at least about 1 micrometer, at least about 2 micrometers, at least about 3 micrometers, at least about 5 micrometers, at least about 10 micrometers, at least about 15 micrometers, or at least about 20 micrometers in certain cases.

The particle may be, in one set of embodiments, a hydrogel particle. See, e.g., Int. Pat. Apl. Pub. No. WO 2008/109176, entitled “Assay and other reactions involving droplets” (incorporated herein by reference) for examples of hydrogel particles, including hydrogel particles containing DNA. Examples of hydrogels include, but are not limited to agarose or acrylamide-based gels, such as polyacrylamide, poly-N-isopropylacrylamide, or poly N-isopropylpolyacrylamide. For example, an aqueous solution of a monomer may be dispersed in a droplet, and then polymerized, e.g., to form a gel. Another example is a hydrogel, such as alginic acid that can be gelled by the addition of calcium ions. In some cases, gelation initiators (ammonium persulfate and TEMED for acrylamide, or Ca²⁺ for alginate) can be added to a droplet, for example, by co-flow with the aqueous phase, by co-flow through the oil phase, or by coalescence of two different drops, e.g., as discussed in U.S. patent application Ser. No. 11/360,845, filed Feb. 23, 2006, entitled “Electronic Control of Fluidic Species,” by Link, et al., published as U.S. Patent Application Publication No. 2007/000342 on Jan. 4, 2007; or in U.S. patent application Ser. No. 11/698,298, filed Jan. 24, 2007, entitled “Fluidic Droplet Coalescence,” by Ahn, et al.; each incorporated herein by reference in their entireties.

In another set of embodiments, the particles may comprise one or more polymers. Exemplary polymers include, but are not limited to, polystyrene (PS), polycaprolactone (PCL), polyisoprene (PIP), poly(lactic acid), polyethylene, polypropylene, polyacrylonitrile, polyimide, polyamide, and/or mixtures and/or co-polymers of these and/or other polymers. In addition, in some cases, the particles may be magnetic, which could allow for the magnetic manipulation of the particles. For example, the particles may comprise iron or other magnetic materials. The particles could also be functionalized so that they could have other molecules attached, such as proteins, nucleic acids or small molecules. Thus, some embodiments of the present invention are directed to a set of particles defining a library of, for example, nucleic acids, proteins, small molecules, or other species such as those described herein. In some embodiments, the particle may be fluorescent.

In some cases, particles such as those discussed herein containing oligonucleotides may be contained within a droplet and the oligonucleotides released from the particle into the interior of the droplet. The droplet may also contain nucleic acid (e.g., produced by lysing a cell), which can be bound to or recognized by the oligonucleotides. The particles and the cells may be introduced within the droplets during and/or after formation of the droplets, and may be added simultaneously or sequentially (in any suitable order). As mentioned, in some embodiments, the particles and the cells may be placed within droplets such that the droplets typically would contain, on average, no more than one particle and no more than one cell.

In some cases, the droplets may be burst, broken, or otherwise disrupted. This may be useful, for example, for subsequent study of the nucleic acids, e.g., via sequencing or other techniques. A wide variety of methods for “breaking” or “bursting” droplets are available to those of ordinary skill in the art, and the exact method chosen is not critical. For example, droplets contained in a carrying fluid may be disrupted using techniques such as mechanical disruption or ultrasound. Droplets may also be disrupted using chemical agents or surfactants, for example, 1H,1H,2H,2H-perfluorooctanol.

Nucleic acids (labeled with oligonucleotides) from different droplets may then be pooled or combined together or analyzed, e.g., sequenced, amplified, etc. The nucleic acids from different droplets, may however, remain distinguishable due to the presence of different oligonucleotides (e.g., containing different barcodes) that were present in each droplet prior to disruption.

For example, the nucleic acids may be amplified using PCR (polymerase chain reaction) or other amplification techniques. Typically, in PCR reactions, the nucleic acids are heated to cause dissociation of the nucleic acids into single strands, and a heat-stable DNA polymerase (such as Taq polymerase) is used to amplify the nucleic acid. This process is often repeated multiple times to amplify the nucleic acids.

In one set of embodiments, the PCR may be used to amplify the nucleic acids. Those of ordinary skill in the art will be aware of suitable PCR techniques and variations, such as assembly PCR or polymerase cycling assembly, which may be used in some embodiments to produce an amplified nucleic acid. Non-limiting examples of such procedures are also discussed below. In addition, in some cases, suitable primers may be used to initiate polymerization, e.g., P5 and P7, or other primers known to those of ordinary skill in the art. Those of ordinary skill in the art will be aware of suitable primers, many of which can be readily obtained commercially.

Other non-limiting examples of amplification methods known to those of ordinary skill in the art that may be used include, but are not limited to, reverse transcriptase (RT) PCR amplification, in vitro transcription amplification (IVT), multiple displacement amplification (MDA), or quantitative real-time PCR (qPCR).

In some embodiments, the nucleic acids may be sequenced using a variety of techniques and instruments, many of which are readily available commercially. Examples of such techniques include, but are not limited to, chain-termination sequencing, sequencing-by-hybridization, Maxam-Gilbert sequencing, dye-terminator sequencing, chain-termination methods, Massively Parallel Signature Sequencing (Lynx Therapeutics), polony sequencing, pyrosequencing, sequencing by ligation, ion semiconductor sequencing, DNA nanoball sequencing, single-molecule real-time sequencing, nanopore sequencing, microfluidic Sanger sequencing, digital RNA sequencing (“digital RNA-seq”), etc. The exact sequencing method chosen is not critical.

In addition, in some cases, the droplets may also contain one or more DNA-tagged antibodies, e.g., to determine proteins in the cell, e.g., by suitable tagging with DNA. Thus, for example, a protein may be detected in a plurality of cells as discussed herein, using DNA-tagged antibodies specific for the protein.

Additional details regarding systems and methods for manipulating droplets in a microfluidic system in accordance with various aspects of the invention follow, e.g., for determining droplets (or species within droplets), sorting droplets, etc. For example, various systems and methods for screening and/or sorting droplets are described in U.S. patent application Ser. No. 11/360,845, filed Feb. 23, 2006, entitled “Electronic Control of Fluidic Species,” by Link, et al., published as U.S. Patent Application Publication No. 2007/000342 on Jan. 4, 2007, incorporated herein by reference. As a non-limiting example, by applying (or removing) a first electric field (or a portion thereof), a droplet may be directed to a first region or channel; by applying (or removing) a second electric field to the device (or a portion thereof), the droplet may be directed to a second region or channel; by applying a third electric field to the device (or a portion thereof), the droplet may be directed to a third region or channel; etc., where the electric fields may differ in some way, for example, in intensity, direction, frequency, duration, etc.

In certain embodiments of the invention, sensors are provided that can sense and/or determine one or more characteristics of the fluidic droplets, and/or a characteristic of a portion of the fluidic system containing the fluidic droplet (e.g., the liquid surrounding the fluidic droplet) in such a manner as to allow the determination of one or more characteristics of the fluidic droplets. Characteristics determinable with respect to the droplet and usable in the invention can be identified by those of ordinary skill in the art. Non-limiting examples of such characteristics include fluorescence, spectroscopy (e.g., optical, infrared, ultraviolet, etc.), radioactivity, mass, volume, density, temperature, viscosity, pH, concentration of a substance, such as a biological substance (e.g., a protein, a nucleic acid, etc.), or the like.

In some cases, the sensor may be connected to a processor, which in turn, cause an operation to be performed on the fluidic droplet, for example, by sorting the droplet, adding or removing electric charge from the droplet, fusing the droplet with another droplet, splitting the droplet, causing mixing to occur within the droplet, etc., for example, as previously described. For instance, in response to a sensor measurement of a fluidic droplet, a processor may cause the fluidic droplet to be split, merged with a second fluidic droplet, etc.

One or more sensors and/or processors may be positioned to be in sensing communication with the fluidic droplet. “Sensing communication,” as used herein, means that the sensor may be positioned anywhere such that the fluidic droplet within the fluidic system (e.g., within a channel), and/or a portion of the fluidic system containing the fluidic droplet may be sensed and/or determined in some fashion. For example, the sensor may be in sensing communication with the fluidic droplet and/or the portion of the fluidic system containing the fluidic droplet fluidly, optically or visually, thermally, pneumatically, electronically, or the like. The sensor can be positioned proximate the fluidic system, for example, embedded within or integrally connected to a wall of a channel, or positioned separately from the fluidic system but with physical, electrical, and/or optical communication with the fluidic system so as to be able to sense and/or determine the fluidic droplet and/or a portion of the fluidic system containing the fluidic droplet (e.g., a channel or a microchannel, a liquid containing the fluidic droplet, etc.). For example, a sensor may be free of any physical connection with a channel containing a droplet, but may be positioned so as to detect electromagnetic radiation arising from the droplet or the fluidic system, such as infrared, ultraviolet, or visible light. The electromagnetic radiation may be produced by the droplet, and/or may arise from other portions of the fluidic system (or externally of the fluidic system) and interact with the fluidic droplet and/or the portion of the fluidic system containing the fluidic droplet in such as a manner as to indicate one or more characteristics of the fluidic droplet, for example, through absorption, reflection, diffraction, refraction, fluorescence, phosphorescence, changes in polarity, phase changes, changes with respect to time, etc. As an example, a laser may be directed towards the fluidic droplet and/or the liquid surrounding the fluidic droplet, and the fluorescence of the fluidic droplet and/or the surrounding liquid may be determined. “Sensing communication,” as used herein may also be direct or indirect. As an example, light from the fluidic droplet may be directed to a sensor, or directed first through a fiber optic system, a waveguide, etc., before being directed to a sensor.

Non-limiting examples of sensors useful in the invention include optical or electromagnetically-based systems. For example, the sensor may be a fluorescence sensor (e.g., stimulated by a laser), a microscopy system (which may include a camera or other recording device), or the like. As another example, the sensor may be an electronic sensor, e.g., a sensor able to determine an electric field or other electrical characteristic. For example, the sensor may detect capacitance, inductance, etc., of a fluidic droplet and/or the portion of the fluidic system containing the fluidic droplet.

As used herein, a “processor” or a “microprocessor” is any component or device able to receive a signal from one or more sensors, store the signal, and/or direct one or more responses (e.g., as described above), for example, by using a mathematical formula or an electronic or computational circuit. The signal may be any suitable signal indicative of the environmental factor determined by the sensor, for example a pneumatic signal, an electronic signal, an optical signal, a mechanical signal, etc.

In one set of embodiments, a fluidic droplet may be directed by creating an electric charge and/or an electric dipole on the droplet, and steering the droplet using an applied electric field, which may be an AC field, a DC field, etc. As an example, an electric field may be selectively applied and removed (or a different electric field may be applied, e.g., a reversed electric field) as needed to direct the fluidic droplet to a particular region. The electric field may be selectively applied and removed as needed, in some embodiments, without substantially altering the flow of the liquid containing the fluidic droplet. For example, a liquid may flow on a substantially steady-state basis (i.e., the average flowrate of the liquid containing the fluidic droplet deviates by less than 20% or less than 15% of the steady-state flow or the expected value of the flow of liquid with respect to time, and in some cases, the average flowrate may deviate less than 10% or less than 5%) or other predetermined basis through a fluidic system of the invention (e.g., through a channel or a microchannel), and fluidic droplets contained within the liquid may be directed to various regions, e.g., using an electric field, without substantially altering the flow of the liquid through the fluidic system.

In some embodiments, the fluidic droplets may be screened or sorted within a fluidic system of the invention by altering the flow of the liquid containing the droplets. For instance, in one set of embodiments, a fluidic droplet may be steered or sorted by directing the liquid surrounding the fluidic droplet into a first channel, a second channel, etc.

In another set of embodiments, pressure within a fluidic system, for example, within different channels or within different portions of a channel, can be controlled to direct the flow of fluidic droplets. For example, a droplet can be directed toward a channel junction including multiple options for further direction of flow (e.g., directed toward a branch, or fork, in a channel defining optional downstream flow channels). Pressure within one or more of the optional downstream flow channels can be controlled to direct the droplet selectively into one of the channels, and changes in pressure can be effected on the order of the time required for successive droplets to reach the junction, such that the downstream flow path of each successive droplet can be independently controlled. In one arrangement, the expansion and/or contraction of liquid reservoirs may be used to steer or sort a fluidic droplet into a channel, e.g., by causing directed movement of the liquid containing the fluidic droplet. The liquid reservoirs may be positioned such that, when activated, the movement of liquid caused by the activated reservoirs causes the liquid to flow in a preferred direction, carrying the fluidic droplet in that preferred direction. For instance, the expansion of a liquid reservoir may cause a flow of liquid towards the reservoir, while the contraction of a liquid reservoir may cause a flow of liquid away from the reservoir. In some cases, the expansion and/or contraction of the liquid reservoir may be combined with other flow-controlling devices and methods, e.g., as described herein. Non-limiting examples of devices able to cause the expansion and/or contraction of a liquid reservoir include pistons and piezoelectric components. In some cases, piezoelectric components may be particularly useful due to their relatively rapid response times, e.g., in response to an electrical signal. In some embodiments, the fluidic droplets may be sorted into more than two channels.

As mentioned, certain embodiments are generally directed to systems and methods for sorting fluidic droplets in a liquid, and in some cases, at relatively high rates. For example, a property of a droplet may be sensed and/or determined in some fashion (e.g., as further described herein), then the droplet may be directed towards a particular region of the device, such as a microfluidic channel, for example, for sorting purposes. In some cases, high sorting speeds may be achievable using certain systems and methods of the invention. For instance, at least about 10 droplets per second may be determined and/or sorted in some cases, and in other cases, at least about 20 droplets per second, at least about 30 droplets per second, at least about 100 droplets per second, at least about 200 droplets per second, at least about 300 droplets per second, at least about 500 droplets per second, at least about 750 droplets per second, at least about 1,000 droplets per second, at least about 1,500 droplets per second, at least about 2,000 droplets per second, at least about 3,000 droplets per second, at least about 5,000 droplets per second, at least about 7,500 droplets per second, at least about 10,000 droplets per second, at least about 15,000 droplets per second, at least about 20,000 droplets per second, at least about 30,000 droplets per second, at least about 50,000 droplets per second, at least about 75,000 droplets per second, at least about 100,000 droplets per second, at least about 150,000 droplets per second, at least about 200,000 droplets per second, at least about 300,000 droplets per second, at least about 500,000 droplets per second, at least about 750,000 droplets per second, at least about 1,000,000 droplets per second, at least about 1,500,000 droplets per second, at least about 2,000,000 or more droplets per second, or at least about 3,000,000 or more droplets per second may be determined and/or sorted.

In some aspects, a population of relatively small droplets may be used. In certain embodiments, as non-limiting examples, the average diameter of the droplets may be less than about 1 mm, less than about 500 micrometers, less than about 300 micrometers, less than about 200 micrometers, less than about 100 micrometers, less than about 75 micrometers, less than about 50 micrometers, less than about 30 micrometers, less than about 25 micrometers, less than about 20 micrometers, less than about 15 micrometers, less than about 10 micrometers, less than about 5 micrometers, less than about 3 micrometers, less than about 2 micrometers, less than about 1 micrometer, less than about 500 nm, less than about 300 nm, less than about 100 nm, or less than about 50 nm. The average diameter of the droplets may also be at least about 30 nm, at least about 50 nm, at least about 100 nm, at least about 300 nm, at least about 500 nm, at least about 1 micrometer, at least about 2 micrometers, at least about 3 micrometers, at least about 5 micrometers, at least about 10 micrometers, at least about 15 micrometers, or at least about 20 micrometers in certain cases. The “average diameter” of a population of droplets is the arithmetic average of the diameters of the droplets.

In some embodiments, the droplets may be of substantially the same shape and/or size (i.e., “monodisperse”), or of different shapes and/or sizes, depending on the particular application. In some cases, the droplets may have a homogenous distribution of cross-sectional diameters, i.e., the droplets may have a distribution of diameters such that no more than about 5%, no more than about 2%, or no more than about 1% of the droplets have a diameter less than about 90% (or less than about 95%, or less than about 99%) and/or greater than about 110% (or greater than about 105%, or greater than about 101%) of the overall average diameter of the plurality of droplets. Some techniques for producing homogenous distributions of cross-sectional diameters of droplets are disclosed in International Patent Application No. PCT/US2004/010903, filed Apr. 9, 2004, entitled “Formation and Control of Fluidic Species,” by Link et al., published as WO 2004/091763 on Oct. 28, 2004, incorporated herein by reference.

Those of ordinary skill in the art will be able to determine the average diameter of a population of droplets, for example, using laser light scattering or other known techniques. The droplets so formed can be spherical, or non-spherical in certain cases. The diameter of a droplet, in a non-spherical droplet, may be taken as the diameter of a perfect mathematical sphere having the same volume as the non-spherical droplet.

In some embodiments, one or more droplets may be created within a channel by creating an electric charge on a fluid surrounded by a liquid, which may cause the fluid to separate into individual droplets within the liquid. In some embodiments, an electric field may be applied to the fluid to cause droplet formation to occur. The fluid can be present as a series of individual charged and/or electrically inducible droplets within the liquid. Electric charge may be created in the fluid within the liquid using any suitable technique, for example, by placing the fluid within an electric field (which may be AC, DC, etc.), and/or causing a reaction to occur that causes the fluid to have an electric charge.

The electric field, in some embodiments, is generated from an electric field generator, i.e., a device or system able to create an electric field that can be applied to the fluid. The electric field generator may produce an AC field (i.e., one that varies periodically with respect to time, for example, sinusoidally, sawtooth, square, etc.), a DC field (i.e., one that is constant with respect to time), a pulsed field, etc. Techniques for producing a suitable electric field (which may be AC, DC, etc.) are known to those of ordinary skill in the art. For example, in one embodiment, an electric field is produced by applying voltage across a pair of electrodes, which may be positioned proximate a channel such that at least a portion of the electric field interacts with the channel. The electrodes can be fashioned from any suitable electrode material or materials known to those of ordinary skill in the art, including, but not limited to, silver, gold, copper, carbon, platinum, copper, tungsten, tin, cadmium, nickel, indium tin oxide (“ITO”), etc., as well as combinations thereof.

In another set of embodiments, droplets of fluid can be created from a fluid surrounded by a liquid within a channel by altering the channel dimensions in a manner that is able to induce the fluid to form individual droplets. The channel may, for example, be a channel that expands relative to the direction of flow, e.g., such that the fluid does not adhere to the channel walls and forms individual droplets instead, or a channel that narrows relative to the direction of flow, e.g., such that the fluid is forced to coalesce into individual droplets. In some cases, the channel dimensions may be altered with respect to time (for example, mechanically or electromechanically, pneumatically, etc.) in such a manner as to cause the formation of individual droplets to occur. For example, the channel may be mechanically contracted (“squeezed”) to cause droplet formation, or a fluid stream may be mechanically disrupted to cause droplet formation, for example, through the use of moving baffles, rotating blades, or the like. Other techniques of creating droplets include, for example mixing or vortexing of a fluid.

Certain embodiments are generally directed to systems and methods for splitting a droplet into two or more droplets. For example, a droplet can be split using an applied electric field. The droplet may have a greater electrical conductivity than the surrounding liquid, and, in some cases, the droplet may be neutrally charged. In certain embodiments, in an applied electric field, electric charge may be urged to migrate from the interior of the droplet to the surface to be distributed thereon, which may thereby cancel the electric field experienced in the interior of the droplet. In some embodiments, the electric charge on the surface of the droplet may also experience a force due to the applied electric field, which causes charges having opposite polarities to migrate in opposite directions. The charge migration may, in some cases, cause the drop to be pulled apart into two separate droplets.

Some embodiments of the invention generally relate to systems and methods for fusing or coalescing two or more droplets into one droplet, e.g., where the two or more droplets ordinarily are unable to fuse or coalesce, for example, due to composition, surface tension, droplet size, the presence or absence of surfactants, etc. In certain cases, the surface tension of the droplets, relative to the size of the droplets, may also prevent fusion or coalescence of the droplets from occurring.

As a non-limiting example, two droplets can be given opposite electric charges (i.e., positive and negative charges, not necessarily of the same magnitude), which can increase the electrical interaction of the two droplets such that fusion or coalescence of the droplets can occur due to their opposite electric charges. For instance, an electric field may be applied to the droplets, the droplets may be passed through a capacitor, a chemical reaction may cause the droplets to become charged, etc. The droplets, in some cases, may not be able to fuse even if a surfactant is applied to lower the surface tension of the droplets. However, if the droplets are electrically charged with opposite charges (which can be, but are not necessarily of, the same magnitude), the droplets may be able to fuse or coalesce. As another example, the droplets may not necessarily be given opposite electric charges (and, in some cases, may not be given any electric charge), and are fused through the use of dipoles induced in the droplets that causes the droplets to coalesce. Also, the two or more droplets allowed to coalesce are not necessarily required to meet “head-on.” Any angle of contact, so long as at least some fusion of the droplets initially occurs, is sufficient. See also, e.g., U.S. patent application Ser. No. 11/698,298, filed Jan. 24, 2007, entitled “Fluidic Droplet Coalescence,” by Ahn, et al., published as U.S. Patent Application Publication No. 2007/0195127 on Aug. 23, 2007, incorporated herein by reference in its entirety.

In one set of embodiments, a fluid may be injected into a droplet. The fluid may be microinjected into the droplet in some cases, e.g., using a microneedle or other such device. In other cases, the fluid may be injected directly into a droplet using a fluidic channel as the droplet comes into contact with the fluidic channel. Other techniques of fluid injection are disclosed in, e.g., International Patent Application No. PCT/US2010/040006, filed Jun. 25, 2010, entitled “Fluid Injection,” by Weitz, et al., published as WO 2010/151776 on Dec. 29, 2010; or International Patent Application No. PCT/US2009/006649, filed Dec. 18, 2009, entitled “Particle-Assisted Nucleic Acid Sequencing,” by Weitz, et al., published as WO 2010/080134 on Jul. 15, 2010, each incorporated herein by reference in its entirety.

A variety of materials and methods, according to certain aspects of the invention, can be used to form articles or components such as those described herein, e.g., channels such as microfluidic channels, chambers, etc. For example, various articles or components can be formed from solid materials, in which the channels can be formed via micromachining, film deposition processes such as spin coating and chemical vapor deposition, laser fabrication, photolithographic techniques, etching methods including wet chemical or plasma processes, and the like. See, for example, Scientific American, 248:44-55, 1983 (Angell, et al).

In one set of embodiments, various structures or components of the articles described herein can be formed of a polymer, for example, an elastomeric polymer such as polydimethylsiloxane (“PDMS”), polytetrafluoroethylene (“PTFE” or Teflon®), or the like. For instance, according to one embodiment, a microfluidic channel may be implemented by fabricating the fluidic system separately using PDMS or other soft lithography techniques (details of soft lithography techniques suitable for this embodiment are discussed in the references entitled “Soft Lithography,” by Younan Xia and George M. Whitesides, published in the Annual Review of Material Science, 1998, Vol. 28, pages 153-184, and “Soft Lithography in Biology and Biochemistry,” by George M. Whitesides, Emanuele Ostuni, Shuichi Takayama, Xingyu Jiang and Donald E. Ingber, published in the Annual Review of Biomedical Engineering, 2001, Vol. 3, pages 335-373; each of these references is incorporated herein by reference).

Other examples of potentially suitable polymers include, but are not limited to, polyethylene terephthalate (PET), polyacrylate, polymethacrylate, polycarbonate, polystyrene, polyethylene, polypropylene, polyvinylchloride, cyclic olefin copolymer (COC), polytetrafluoroethylene, a fluorinated polymer, a silicone such as polydimethylsiloxane, polyvinylidene chloride, bis-benzocyclobutene (“BCB”), a polyimide, a fluorinated derivative of a polyimide, or the like. Combinations, copolymers, or blends involving polymers including those described above are also envisioned. The device may also be formed from composite materials, for example, a composite of a polymer and a semiconductor material.

In some embodiments, various structures or components of the article are fabricated from polymeric and/or flexible and/or elastomeric materials, and can be conveniently formed of a hardenable fluid, facilitating fabrication via molding (e.g. replica molding, injection molding, cast molding, etc.). The hardenable fluid can be essentially any fluid that can be induced to solidify, or that spontaneously solidifies, into a solid capable of containing and/or transporting fluids contemplated for use in and with the fluidic network. In one embodiment, the hardenable fluid comprises a polymeric liquid or a liquid polymeric precursor (i.e. a “prepolymer”). Suitable polymeric liquids can include, for example, thermoplastic polymers, thermoset polymers, waxes, metals, or mixtures or composites thereof heated above their melting point. As another example, a suitable polymeric liquid may include a solution of one or more polymers in a suitable solvent, which solution forms a solid polymeric material upon removal of the solvent, for example, by evaporation. Such polymeric materials, which can be solidified from, for example, a melt state or by solvent evaporation, are well known to those of ordinary skill in the art. A variety of polymeric materials, many of which are elastomeric, are suitable, and are also suitable for forming molds or mold masters, for embodiments where one or both of the mold masters is composed of an elastomeric material. A non-limiting list of examples of such polymers includes polymers of the general classes of silicone polymers, epoxy polymers, and acrylate polymers. Epoxy polymers are characterized by the presence of a three-membered cyclic ether group commonly referred to as an epoxy group, 1,2-epoxide, or oxirane. For example, diglycidyl ethers of bisphenol A can be used, in addition to compounds based on aromatic amine, triazine, and cycloaliphatic backbones. Another example includes the well-known Novolac polymers. Non-limiting examples of silicone elastomers suitable for use according to the invention include those formed from precursors including the chlorosilanes such as methylchlorosilanes, ethylchlorosilanes, phenylchlorosilanes, dodecyltrichlorosilanes, etc.

Silicone polymers are used in certain embodiments, for example, the silicone elastomer polydimethylsiloxane. Non-limiting examples of PDMS polymers include those sold under the trademark Sylgard by Dow Chemical Co., Midland, Mich., and particularly Sylgard 182, Sylgard 184, and Sylgard 186. Silicone polymers including PDMS have several beneficial properties simplifying fabrication of various structures of the invention. For instance, such materials are inexpensive, readily available, and can be solidified from a prepolymeric liquid via curing with heat. For example, PDMSs are typically curable by exposure of the prepolymeric liquid to temperatures of about, for example, about 65° C. to about 75° C. for exposure times of, for example, about an hour. Also, silicone polymers, such as PDMS, can be elastomeric and thus may be useful for forming very small features with relatively high aspect ratios, necessary in certain embodiments of the invention. Flexible (e.g., elastomeric) molds or masters can be advantageous in this regard.

One advantage of forming structures such as microfluidic structures or channels from silicone polymers, such as PDMS, is the ability of such polymers to be oxidized, for example by exposure to an oxygen-containing plasma such as an air plasma, so that the oxidized structures contain, at their surface, chemical groups capable of cross-linking to other oxidized silicone polymer surfaces or to the oxidized surfaces of a variety of other polymeric and non-polymeric materials. Thus, structures can be fabricated and then oxidized and essentially irreversibly sealed to other silicone polymer surfaces, or to the surfaces of other substrates reactive with the oxidized silicone polymer surfaces, without the need for separate adhesives or other sealing means. In most cases, sealing can be completed simply by contacting an oxidized silicone surface to another surface without the need to apply auxiliary pressure to form the seal. That is, the pre-oxidized silicone surface acts as a contact adhesive against suitable mating surfaces. Specifically, in addition to being irreversibly sealable to itself, oxidized silicone such as oxidized PDMS can also be sealed irreversibly to a range of oxidized materials other than itself including, for example, glass, silicon, silicon oxide, quartz, silicon nitride, polyethylene, polystyrene, glassy carbon, and epoxy polymers, which have been oxidized in a similar fashion to the PDMS surface (for example, via exposure to an oxygen-containing plasma). Oxidation and sealing methods useful in the context of the present invention, as well as overall molding techniques, are described in the art, for example, in an article entitled “Rapid Prototyping of Microfluidic Systems and Polydimethylsiloxane,” Anal. Chem., 70:474-480, 1998 (Duffy et al.), incorporated herein by reference.

Thus, in certain embodiments, the design and/or fabrication of the article may be relatively simple, e.g., by using relatively well-known soft lithography and other techniques such as those described herein. In addition, in some embodiments, rapid and/or customized design of the article is possible, for example, in terms of geometry. In one set of embodiments, the article may be produced to be disposable, for example, in embodiments where the article is used with substances that are radioactive, toxic, poisonous, reactive, biohazardous, etc., and/or where the profile of the substance (e.g., the toxicology profile, the radioactivity profile, etc.) is unknown. Another advantage to forming channels or other structures (or interior, fluid-contacting surfaces) from oxidized silicone polymers is that these surfaces can be much more hydrophilic than the surfaces of typical elastomeric polymers (where a hydrophilic interior surface is desired). Such hydrophilic channel surfaces can thus be more easily filled and wetted with aqueous solutions than can structures comprised of typical, unoxidized elastomeric polymers or other hydrophobic materials.

The following documents are each incorporated herein by reference in its entirety for all purposes: U.S. Pat. Apl. Ser. No. 61/980,541, entitled “Methods and Systems for Droplet Tagging and Amplification,” by Weitz, et al.; U.S. Pat. Apl. Ser. No. 61/981,123, entitled “Systems and Methods for Droplet Tagging,” by Bernstein, et al.; Int. Pat. Apl. Pub. No. WO 2004/091763, entitled “Formation and Control of Fluidic Species,” by Link et al.; Int. Pat. Apl. Pub. No. WO 2004/002627, entitled “Method and Apparatus for Fluid Dispersion,” by Stone et al.; Int. Pat. Apl. Pub. No. WO 2006/096571, entitled “Method and Apparatus for Forming Multiple Emulsions,” by Weitz et al.; Int. Pat. Apl. Pub. No. WO 2005/021151, entitled “Electronic Control of Fluidic Species,” by Link et al.; Int. Pat. Apl. Pub. No. WO 2011/056546, entitled “Droplet Creation Techniques,” by Weitz, et al.; Int. Pat. Apl. Pub. No. WO 2010/033200, entitled “Creation of Libraries of Droplets and Related Species,” by Weitz, et al.; U.S. Pat. Apl. Pub. No. 2012-0132288, entitled “Fluid Injection,” by Weitz, et al.; Int. Pat. Apl. Pub. No. WO 2008/109176, entitled “Assay And Other Reactions Involving Droplets,” by Agresti, et al.; and Int. Pat. Apl. Pub. No. WO 2010/151776, entitled “Fluid Injection,” by Weitz, et al.; Int. Pat. Apl. Pub. No. WO 2015/164212, entitled “Systems and Methods for Barcoding Nucleic Acids,” by Weitz, et al.; Int. Pa. Apl. Pub. No. WO 2015/16122, entitled “Methods and Systems for Droplet Tagging and Amplification”; and Int. Pat. Apl. Pub. No. WO 2015/16117, entitled “Systems and Methods for Droplet Tagging.”

In addition, the following are incorporated herein by reference in their entireties: U.S. Pat. Apl. Ser. No. 61/981,123, filed Apr. 17, 2014, entitled “Systems and Methods for Droplet Tagging”; Int. Pat. Apl. Ser. No. PCT/US2015/026338, filed Apr. 17, 2015, entitled “Systems and Methods for Droplet Tagging”; U.S. Pat. Apl. Ser. No. 61/981,108 filed Apr. 17, 2014, entitled “Methods and Systems for Droplet Tagging and Amplification”; Int. Pat. Apl. Ser. No. PCT/US15/26422, filed on Apr. 17, 2015, entitled “Methods and Systems for Droplet Tagging and Amplification”; U.S. Pat. Apl. Ser. No. 62/149,372, filed on Apr. 17, 2015, entitled “Immobilization-Based Systems and Methods for Genetic Analysis and Other Applications”; U.S. Pat. Apl. Ser. No. 62/072,944, filed Oct. 30, 2014, entitled “Systems and Methods for Barcoding Nucleic Acids”; Int. Pat. Apl. Ser. No. PCT/US15/26443, filed on Apr. 17, 2015, entitled “Systems and Methods for Barcoding Nucleic Acids”; and U.S. Pat. Apl. Ser. No. 62/365,278, filed Jul. 21, 2016, entitled “Microfluidic Sequencing Techniques,” by Weitz, et al.

The following examples are intended to illustrate certain embodiments of the present invention, but do not exemplify the full scope of the invention.

### Example 1

This example illustrates a technique to sequence mutations in rare cells. In this example, a special forward primer is used to introduce a restriction site into PCR amplicons if the template is wild-type. After incubation with a certain restriction enzyme the amplicons carrying mutations are digested. However, if the template is mutant the corresponding restriction site cannot be generated, such that mutant amplicons cannot be digested. Therefore, the amplicons from the rare mutant cells are selectively sequenced, as shown in FIG. 1. The frequency of mutant cells can be estimated using the number of mutant cells based on counting sequenced cells, divided the total number of cells based on encapsulation.

Single cells are encapsulated with a mixture of lysis buffer and RT-PCR/PCR reagent in a water-in-oil emulsion. Besides polymerase and buffer, the RT-PCR/PCR reagent includes a pool of primers pairs for multiplexing amplification in which the forward primers can introduce restriction sites during amplification, followed by RT-PCR/PCR. Non-limiting examples of primers and restriction enzymes are listed in Table 1. In this table, the lower case letters indicate bases that are not complementarily paired with the target.

The drops are injected into a microfluidic picoinjector (see, e.g., U.S. Pat. Apl. Pub. No. 2012-0132288, incorporated herein by reference in its entirety) and the droplets spaced with oil containing surfactant. Downstream of the picoinjector, DNA-barcoded hydrogel beads are electrically injected into the drops, together with the second PCR mixture to introduce the barcodes on the hydrogel beads into the original PCR amplicons. After amplification in drops, the drops are broken by adding a drop destabilizer, such as 1H, 1H, 2H, 2H-perfluoro-octanol. The droplets are briefly vortexed and centrifuged. To deplete the wild-type amplicons, a mixture of restriction enzymes is added with incubation at 37° C. for 2 hours. A DNA gel purification is then performed to extract the amplicons, followed by PCR to introduce indices or nucleic acid tags, which allows the samples to be multiplexed. Finally, the amplicons are sequenced and bioinformatics used to analyze the results to obtain mutation information on the cells.

TABLE 1

| | | |
|---|---|---|
| BTK C481S | Forward primer | CAGGAGUGAGAUGACAGGAGGCCCCAU |
| | | CTTCATCAUCACTGAGUtatcacacagUGGCT |
| | | G |
| | | (SEQ ID NO: 1) |
| | Reverse primer | GUCTCGUGGGCUCGGAGAUGTGTAUAA |
| | | GAGACAGacgCACAGACAUCCTUGCACAT |
| | | CUCTA |
| | | (SEQ ID NO: 2) |
| | Restriction enzyme | AlwNI |
| PLCG2 | Forward primer | CAGGAGUGAGAUGACAGGAGCACGACG |
| L845F | | UTATAGGUATTGAGGUCCAAUtactuGAGA |
| | | CCCU |
| | | (SEQ ID NO: 3) |
| | Reverse primer | GUCTCGUGGGCUCGGAGAUGTGTAUAA |
| | | GAGACAG |
| | | cgaAACUACGUCGAGGACATCUCAA |
| | | (SEQ ID NO: 4) |
| | Restriction enzyme | Bsu36I |
| PLCG2 | Forward primer | CAGGAGUGAGAUGACAGGAGGCGGAGA |
| R665W | | GGCAGAGGACAauactucATUCCCC |
| | | (SEQ ID NO: 5) |
| | Reverse primer | GUCTCGUGGGCUCGGAGAUGTGTAUAA |
| | | GAGACAGgcaUGATGGCAUAGGAGUCGC |
| | | U |
| | | (SEQ ID NO: 6) |
| | Restriction enzyme | SmaI |
| PLCG2 | Forward primer | CAGGAGUGAGAUGACAGGAGCGUAGTA |
| S707Y | | ACUGACGAGCUCCACCUTCCTUTAGGCG |
| | | G |
| | | (SEQ ID NO: 7) |
| | Reverse primer | GUCTCGUGGGCUCGGAGAUGTGTAUAA |
| | | GAGACAGccaGCAAGGUAAAGCAUTGUC |
| | | GCA |
| | | (SEQ ID NO: 8) |
| | Restriction enzyme | BslI |

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the invention includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
lysing cells contained within microfluidic droplets to release nucleic acids;
amplifying the released nucleic acids within the droplets using primers that introduce restriction sites during amplification to produce amplicons;
bonding nucleic acid tags to at least some of the amplicons within the droplets;
releasing the amplicons from the droplets;
exposing the amplicons to a restriction enzyme; and
sequencing the amplicons.

2. The method of claim 1, wherein the cells comprise mammalian cells.

3. The method of any one of claim 1 or 2, wherein the cells comprise human cells.

4. The method of any one of claims 1-3, wherein the released nucleic acids comprise genomic DNA.

5. The method of any one of claims 1-4, wherein the restriction site introduced by the primer is cleavable by the restriction enzyme.

6. The method of any one of claims 1-5, wherein the restriction enzyme is EcoRI.

7. The method of any one of claims 1-5, wherein the restriction enzyme is AlwNI.

8. The method of any one of claims 1-5, wherein the restriction enzyme is Bsu36I.

9. The method of any one of claims 1-5, wherein the restriction enzyme is SmaI.

10. The method of any one of claims 1-10, wherein the restriction enzyme is BslI.

11. The method of any one of claims 1-5, wherein the sequence cleavable by the restriction enzyme is GAATTC (SEQ ID NO: 9).

12. The method of any one of claims 1-5, wherein the sequence cleavable by the restriction enzyme is CAGNNNCTG (SEQ ID NO: 10).

13. The method of any one of claims 1-5, wherein the sequence cleavable by the restriction enzyme is CCTNAGG (SEQ ID NO: 11).

14. The method of any one of claims 1-5, wherein the sequence cleavable by the restriction enzyme is CCCGGG (SEQ ID NO: 12).

15. The method of any one of claims 1-5, wherein the sequence cleavable by the restriction enzyme is CCNNNNNNNGG (SEQ ID NO: 13).

16. The method of any one of claims 1-15, further comprising:
providing a particle containing nucleic acid tags within the microfluidic droplet; and
cleaving the nucleic acid tags from the particle to release the nucleic acid tags.

17. The method of claim 16, comprising photocleaving the nucleic acid tags from the particle.

18. The method of any one of claim 16 or 17, wherein at least some of the nucleic acid tags are covalently bonded to the particle via an acrylic phosphoramidite linkage.

19. The method of any one of claims 16-18, wherein at least some of the particles are hydrogel particles.

20. The method of any one of claims 16-19, wherein the plurality of particles have an average diameter of no more than about 500 micrometers.

21. The method of any one of claims 1-20, comprising bonding nucleic acid tags to at least some of the amplicons using an enzyme.

22. The method of any one of claims 1-21, wherein the nucleic acid tags uniquely identify the amplicons within the droplets from amplicons contained within other droplets.

23. The method of any one of claims 1-22, wherein the cells are encapsulated within the droplets at no more than about 1 cell/droplet.

24. The method of any one of claims 1-23, wherein the nucleic acid tags are selected from a pool of nucleic acid tags.

25. The method of claim 24, wherein the pool of nucleic acid tags comprises at least 10,000 unique nucleic acid tags.

26. The method of any one of claims 1-25, wherein at least some of the cells are lysed using a cell lysis reagent.

27. The method of any one of claims 1-26, wherein at least some of the cells are lysed using ultrasound.

28. The method of any one of claims 1-27, wherein releasing the amplicons from the droplets comprises breaking the droplets.

29. The method of any one of claims 1-28, wherein the microfluidic droplets have an average diameter of less than about 1 mm.

30. The method of any one of claims 1-29, wherein at least some of the cells arise from dissociated tissue.

31. A method, comprising:
lysing cells contained within microfluidic droplets to release nucleic acids;
amplifying the released nucleic acids within the droplets using primers that introduce restriction sites during amplification to produce amplicons;
bonding nucleic acid tags to at least some of the amplicons within the droplets;
releasing the amplicons from the droplets;
exposing the amplicons to a restriction enzyme; and
determining the amplicons not cleaved by the restriction enzyme.

32. The method of claim 1, wherein the cells comprise mammalian cells.

33. The method of any one of claim 31 or 32, wherein the cells comprise human cells.

34. The method of any one of claims 31-33, wherein the released nucleic acids comprise genomic DNA.

35. The method of any one of claims 31-34, wherein the restriction site introduced by the primer is cleavable by the restriction enzyme.

36. The method of any one of claims 31-35, wherein the restriction enzyme is EcoRI.

37. The method of any one of claims 31-35, wherein the restriction enzyme is AlwNI.

38. The method of any one of claims 31-35, wherein the restriction enzyme is Bsu36I.

39. The method of any one of claims 31-35, wherein the restriction enzyme is SmaI.

40. The method of any one of claims 31-35, wherein the restriction enzyme is BslI.

41. The method of any one of claims 31-35, wherein the sequence cleavable by the restriction enzyme is GAATTC (SEQ ID NO: 9).

42. The method of any one of claims 31-35, wherein the sequence cleavable by the restriction enzyme is CAGNNNCTG (SEQ ID NO: 10).

43. The method of any one of claims 31-35, wherein the sequence cleavable by the restriction enzyme is CCTNAGG (SEQ ID NO: 11).

44. The method of any one of claims 31-35, wherein the sequence cleavable by the restriction enzyme is CCCGGG (SEQ ID NO: 12).

45. The method of any one of claims 31-35, wherein the sequence cleavable by the restriction enzyme is CCNNNNNNNGG (SEQ ID NO: 13).

46. The method of any one of claims 31-45, further comprising:
providing a particle containing nucleic acid tags within the microfluidic droplet; and
cleaving the nucleic acid tags from the particle to release the nucleic acid tags.

47. The method of claim 46, comprising photocleaving the nucleic acid tags from the particle.

48. The method of any one of claim 46 or 47, wherein at least some of the nucleic acid tags are covalently bonded to the particle via an acrylic phosphoramidite linkage.

49. The method of any one of claims 46-48, wherein at least some of the particles are hydrogel particles.

50. The method of any one of claims 46-49, wherein the plurality of particles have an average diameter of no more than about 500 micrometers.

51. The method of any one of claims 31-50, comprising bonding nucleic acid tags to at least some of the amplicons using an enzyme.

52. The method of any one of claims 31-51, wherein the nucleic acid tags uniquely identify the amplicons within the droplets from amplicons contained within other droplets.

53. The method of any one of claims 31-52, wherein the cells are encapsulated within the droplets at no more than about 1 cell/droplet.

54. The method of any one of claims 31-53, wherein the nucleic acid tags are selected from a pool of nucleic acid tags.

55. The method of claim 54, wherein the pool of nucleic acid tags comprises at least 10,000 unique nucleic acid tags.

56. The method of any one of claims 31-55, wherein at least some of the cells are lysed using a cell lysis reagent.

57. The method of any one of claims 31-56, wherein at least some of the cells are lysed using ultrasound.

58. The method of any one of claims 31-57, wherein releasing the amplicons from the droplets comprises breaking the droplets.

59. The method of any one of claims 31-58, wherein the microfluidic droplets have an average diameter of less than about 1 mm.

60. The method of any one of claims 31-59, wherein at least some of the cells arise from dissociated tissue.

61. A method, comprising:
lysing cells contained within microfluidic droplets to release nucleic acids;
amplifying the released nucleic acids within the droplets using primers that introduce restriction sites during amplification to produce amplicons;
releasing the amplicons from the droplets; and
exposing the amplicons to restriction enzymes.

62. The method of claim 61, wherein the cells comprise mammalian cells.

63. The method of any one of claim 61 or 62, wherein the cells comprise human cells.

64. The method of any one of claims 61-63, wherein the released nucleic acids comprise genomic DNA.

65. The method of any one of claims 61-64, wherein the restriction site introduced by the primer is cleavable by the restriction enzyme.

66. The method of any one of claims 61-65, wherein the restriction enzyme is EcoRI.

67. The method of any one of claims 61-65, wherein the restriction enzyme is AlwNI.

68. The method of any one of claims 61-65, wherein the restriction enzyme is Bsu36I.

69. The method of any one of claims 61-65, wherein the restriction enzyme is BslI.

70. The method of any one of claims 61-65, wherein the restriction enzyme is Bsl.

71. The method of any one of claims 61-65, wherein the sequence cleavable by the restriction enzyme is GAATTC (SEQ ID NO: 9).

72. The method of any one of claims 61-65, wherein the sequence cleavable by the restriction enzyme is CAGNNNCTG (SEQ ID NO: 10).

73. The method of any one of claims 61-65, wherein the sequence cleavable by the restriction enzyme is CCTNAGG (SEQ ID NO: 11).

74. The method of any one of claims 61-65, wherein the sequence cleavable by the restriction enzyme is CCCGGG (SEQ ID NO: 12).

75. The method of any one of claims 61-65, wherein the sequence cleavable by the restriction enzyme is CCNNNNNNNGG (SEQ ID NO: 13).

76. The method of any one of claims 61-75, further comprising:
providing a particle containing nucleic acid tags within the microfluidic droplet; and
cleaving the nucleic acid tags from the particle to release the nucleic acid tags.

77. The method of claim 76, comprising photocleaving the nucleic acid tags from the particle.

78. The method of any one of claim 76 or 77, wherein at least some of the nucleic acid tags are covalently bonded to the particle via an acrylic phosphoramidite linkage.

79. The method of any one of claims 76-78, wherein at least some of the particles are hydrogel particles.

80. The method of any one of claims 76-79, wherein the plurality of particles have an average diameter of no more than about 500 micrometers.

81. The method of any one of claims 61-80, comprising bonding nucleic acid tags to at least some of the amplicons using an enzyme.

82. The method of any one of claims 61-81, wherein the nucleic acid tags uniquely identify the amplicons within the droplets from amplicons contained within other droplets.

83. The method of any one of claims 61-82, wherein the cells are encapsulated within the droplets at no more than about 1 cell/droplet.

84. The method of any one of claims 61-83, wherein the nucleic acid tags are selected from a pool of nucleic acid tags.

85. The method of claim 84, wherein the pool of nucleic acid tags comprises at least 10,000 unique nucleic acid tags.

86. The method of any one of claims 61-85, wherein at least some of the cells are lysed using a cell lysis reagent.

87. The method of any one of claims 61-86, wherein at least some of the cells are lysed using ultrasound.

88. The method of any one of claims 61-87, wherein releasing the amplicons from the droplets comprises breaking the droplets.

89. The method of any one of claims 61-88, wherein the microfluidic droplets have an average diameter of less than about 1 mm.

90. The method of any one of claims 61-89, wherein at least some of the cells arise from dissociated tissue.
